(19) United States
(12) Patent Application Publication
(10) Pub. No.: US 2023/0106313 A1
(43) Pub. Date: Apr. 6, 2023

(54) A BACTERIAL COMPOSITION FOR THE TREATMENT OF CANCER

(71) Applicant: UNIVERSITÄT ZÜRICH, Zürich (CH)

(72) Inventors: Michael SCHARL, Aesch (CH); Ana MONTALBAN-ARQUES, Zürich (CH); Lubor BORSIG, Glattfelden (CH)

(73) Assignee: UNIVERSITÄT ZÜRICH, Zürich (CH)

(21) Appl. No.: 17/798,897

(22) PCT Filed: Feb. 11, 2021
(86) PCT No.: PCT/EP2021/053390

§ 371 Date: Aug. 11, 2022

(30) Foreign Application Priority Data
Feb. 12, 2020 (EP) 20157010.8

(51) Int. Cl. A61K 35/742; A61P 35/00; A61K 45/06

(52) U.S. Cl. CPC A61K 35/742; A61P 35/00; A61K 45/06

(57) ABSTRACT

The invention provides a bacterial composition comprising, or consisting of, one or more of the genera of bacteria selected from Anaerostipes, and/or Roseburia, for treatment, or for prevention of recurrence, of cancer. In another aspect, the invention relates to a combination medicament for use in the treatment or the prevention of recurrence of cancer comprising a bacterial composition as specified herein and an antineoplastic treatment, particularly a combination medicament comprising a bacterial composition and a cancer chemotherapy drug, or a bacterial composition as provided herein and a cancer immunotherapy drug.

### BACKGROUND OF THE INVENTION

Checkpoint inhibitor antibodies can lead to durable protective immune response to cancer, despite this, a majority of patients fail to respond to immunotherapy treatment regimes. For example, anti-PD1, anti-PDL1, and/or anti-CTLA4 monoclonal antibodies are only effective in up to a half of melanoma patients, and only about 4-5% of metastatic colorectal cancer (CRC) patients. In addition, these drugs can induce severe, and occasionally life-threatening side-effects.

Gut microbiota have recently emerged as a modulator of immunotherapy and chemotherapeutic agents by means of activation of anti-tumour responses. A dysbiotic gut microbiota characterised by a reduction of Clostridiales bacteria (encompassing butyrate-producing species) has been correlated with increased incidence of CRC and other diseases.

However, there are at present few approaches which can ameliorate the harmful effects of an unbalanced gut microbiome. Interventions such as antibiotics, prebiotics, probiotics and faecal transplants have been used to address this problem, but each has particular limitations and possible side-effects.

Based on the above-mentioned state of the art, the objective of the present invention is to provide improved methods and compositions for cancer treatment. This objective is attained by the subject-matter of the independent claims of the present specification.

The studies described here show that delivering a consortium of Clostridiales bacteria enriched in healthy intestine and not CRC patients is sufficient to trigger anti-tumour immune responses, representing a potential therapeutic approach for the treatment of solid tumours. Oral administration of defined consortia or single strains of butyrate-producing bacteria can prevent and treat cancer in vivo by inducing tumour-specific infiltration and activation of CD8+ T cells. In a direct comparison, bacteria, or a combination of bacteria and anti-PD-1 outperformed anti-PD1 in murine models of colorectal cancer and melanoma, and may thus constitute a novel therapeutic approach in the treatment of solid tumours using bacteria as a stand-alone therapy or in combination with checkpoint modulators or other modalities of cancer immunotherapy.

### SUMMARY OF THE INVENTION

In one aspect, the invention provides an isolated bacterial composition comprising, or consisting of, one or more of the genera of bacteria selected from Anaerostipes, and/or Roseburia, for use as a treatment, or a prophylaxis for cancer.

The bacterial composition for treatment or prevention of recurrence of cancer according to the invention is of particular utility in treatment of an epithelial cell-derived tumour, particularly a cancer selected from lung, breast, brain, prostate, spleen, pancreatic, biliary tract, cervical, ovarian, head and neck, oesophageal, gastric, liver, skin, kidney, bone, testicular, small intestinal, colon or rectal cancer (CRC) or bladder cancer, melanoma or non-melanoma skin cancer or sarcoma.

In another aspect, the invention relates to a combination medicament for use in the treatment or the prevention of recurrence of cancer comprising a bacterial composition as specified herein and an antineoplastic treatment, particularly a combination medicament comprising a bacterial composition and a cancer chemotherapy drug, or a bacterial composition as provided herein and a cancer immunotherapy drug.

### DETAILED DESCRIPTION OF THE INVENTION

Terms and Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

The terms “comprising,” “having,” “containing,” and “including,” and other similar forms, and grammatical equivalents thereof, as used herein, are intended to be equivalent in meaning and to be open ended in that an item or items following any one of these words is not meant to be an exhaustive listing of such item or items, or meant to be limited to only the listed item or items. For example, an article “comprising” components A, B, and C can consist of (i.e., contain only) components A, B, and C, or can contain not only components A, B, and C but also one or more other components. As such, it is intended and understood that “comprises” and similar forms thereof, and grammatical equivalents thereof, include disclosure of embodiments of “consisting essentially of” or “consisting of.”

Where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure.

Reference to “about” a value or parameter herein includes (and describes) variations that are directed to that value or parameter per se. For example, description referring to “about X” includes description of “X.”

As used herein, including in the appended claims, the singular forms “a,” “or,” and “the” include plural referents unless the context clearly dictates otherwise.

Unless defined otherwise, all technical and scientific terms used herein have the same meaning as commonly understood by one of ordinary skill in the art (e.g., in cell culture, molecular genetics, nucleic acid chemistry, hybridization techniques and biochemistry). Standard techniques are used for molecular, genetic and biochemical methods (see generally, Sambrook et al., Molecular Cloning: A Laboratory Manual, 4th ed. (2012) Cold Spring Harbor Laboratory Press, Cold Spring Harbor, N.Y. and Ausubel et al., Short Protocols in Molecular Biology (2002) 5th Ed, John Wiley & Sons, Inc.) and chemical methods.

The term patient in the context of the present specification relates to a human subject.

In the context of the present specification, the term cancer immunotherapy, biological or immunomodulatory therapy is meant to encompass types of cancer treatment that help the immune system to fight cancer. Non-limiting examples of cancer immunotherapy include immune checkpoint inhibitory agents and agonists, T cell transfer therapy, cytokines and their recombinant derivatives, adjuvants, and vaccination with small molecules or cells.

In the context of the present specification, the term checkpoint inhibitory agent or checkpoint inhibitor antibody is meant to encompass a cancer immunotherapy agent, particularly an antibody (or antibody-like molecule) capable of disrupting an inhibitory signalling cascade that limits immune cell activation, known in the art as an immune checkpoint mechanism. The terms checkpoint inhibitory agent or checkpoint inhibitor antibody include, without being limited to, an antibody to CTLA-4 (Uniprot P16410), PD-1 (Uniprot Q15116), PD-L1 (Uniprot Q9NZQ7), B7H3 (CD276; Uniprot Q5ZPR3), VISTA (Uniprot Q9H7M9), TIGIT (UniprotQ495A1), TIM-3 (HAVCR2, Uniprot Q8TDQ0), CD158 (killer cell immunoglobulin-like receptor family), and/or TGF-beta (P01137).

The terms checkpoint inhibitory agent or checkpoint inhibitor antibody, or cancer immunotherapy agent encompass, without being limited to, the clinically available antibody drugs ipilimumab (Bristol-Myers Squibb; CAS No. 477202-00-9), nivolumab (Bristol-Myers Squibb; CAS No 946414-94-4), pembrolizumab (Merck Inc.; CAS No. 1374853-91-4), pidilizumab (CAS No. 1036730-42-3), atezolizumab (Roche AG; CAS No. 1380723-44-3), avelumab (Merck KGaA; CAS No. 1537032-82-8), durvalumab (Astra Zenaca, CAS No. 1428935-60-7), and/or cemiplimab (Sanofi Aventis; CAS No. 1801342-60-8).

In the context of the present specification, the term checkpoint agonist agent or checkpoint agonist antibody is meant to further encompass a cancer immunotherapy agent, particularly but not limited to an antibody (or antibody-like molecule) capable of enhancing an immune cell activation signalling cascade. The term checkpoint agonist agent further encompasses cytokines, recombinant immune stimulatory proteins, vaccines, adjuvants and agonist antibodies that promote immune activation. Non-limiting examples of cytokines known to stimulate immune cell activation include, IL-12, IL-2, IL-15, IL-21 and interferon-alpha. The terms checkpoint agonist agent or checkpoint agonist antibody include but are not limited to an antibody to CD122 (Uniprot P14784) and CD137 (4-1 BB; Uniprot Q07011), ICOS (Uniprot Q9Y6W8), OX40 (GP34, Uniprot P43489), and/or CD40 (Uniprot P25942).

In certain embodiments, the term cancer immunotherapy is meant to encompass immune cell transfer cancer treatments wherein a patient's immune cells are activated or expanded in vitro, and/or genetically modified, for example with the addition of a chimeric antigen receptor, before being infused back into the patient to inhibit neoplastic disease. Non-limiting examples of immune cell transfer therapy include chimeric antigen receptor T lymphocytes, and autologous activated T cells or dendritic cells.

As used herein, the term bacterial composition, synonymous with isolated bacterial composition, refers to a preparation of bacteria, optionally together with a pharmaceutically acceptable carrier. The bacterial composition may be manufactured by methods such as growth in a bacterial fermenter, and manufacturing methods for the bacterial composition may include washing, concentration, filtering, encapsulating, lyophilising, drying, emulsifying steps or other processes. Products used in the manufacturing processes such as culture or washing media, or traces of such products, may form part of the bacterial composition. The composition can be in various forms including, but not limited to, granules, powders, emulsions, suspensions, solutions, gels, dermal absorption systems, capsules or tablets. The bacterial composition can be included in a food product, which may optionally include other nutrients or prebiotics such as dietary fibre.

As used herein, the term pharmaceutically acceptable carrier includes any solvents, dispersion media, coatings, surfactants, antioxidants, preservatives (for example, antifungal agents, or antibacterial agents, with the caveat that antibacterial agents are selected, or combined with the bacterial preparation in such a way as to prevent inhibition of their growth, engraftment, or viability, of the constituent bacteria, or that antibacterial agents are not present in embodiments of the invention which require the absence of such agents), isotonic agents, absorption delaying agents, salts, preservatives, drugs, drug stabilizers, binders, excipients, disintegration agents, lubricants, sweetening agents, flavouring agents, dyes, and the like and combinations thereof, as would be known to those skilled in the art (see, for example, Remington: the Science and Practice of Pharmacy, ISBN 0857110624).

As used herein, the term treating or treatment of any disease or disorder (e.g. cancer) refers in one embodiment, to ameliorating the disease or disorder (e.g. slowing or arresting or reducing the development of the disease or at least one of the clinical symptoms thereof). In another embodiment “treating” or “treatment” refers to alleviating or ameliorating at least one physical parameter including those which may not be discernible by the patient. In yet another embodiment, “treating” or “treatment” refers to modulating the disease or disorder, either physically, (e.g., stabilization of a discernible symptom), physiologically, (e.g., stabilization of a physical parameter), or both. Methods for assessing treatment and/or prevention of disease are generally known in the art, unless specifically described hereinbelow.

An aspect of the invention relates to a bacterial composition for treatment, or for prevention of recurrence, of cancer. The composition according to the invention comprises, or in certain embodiments consists of, one or more of the bacterial genera Anaerostipes and/or Roseburia.

In certain particular embodiments, the bacterial composition for treatment or prevention of recurrence of cancer consists of bacteria belonging only to the genera Anaerostipes and/or Roseburia. The term “consists” in this context is exclusive only with regard to the type of bacteria, it relates to the presence of bacteria, in other words, no detectable amounts of bacteria not belonging to either genus are present. The composition may, however, contain excipients, matter for the bacteria to grow on or to be used as substrate of the bacteria when administered to the colon, or other ingredients.

In certain particular embodiments, the bacterial composition for treatment or prevention of recurrence of cancer comprises only bacteria of the genus Anaerostipes, in other words, the composition consists of, as far as the bacterial content is concerned, bacteria of the genus Anaerostipes.

The data in the examples show that a preparation of Anaerostipes alone can offer equal or better protection from cancer progression compared to a mix of different butyrate-producing bacteria previously shown to be reduced in patients with colorectal cancer.

In certain particular embodiments, the bacterial composition for treatment or prevention of recurrence of cancer comprises only bacteria of the genus Roseburia, in other words, the composition consists of, as far as the bacterial content is concerned, bacteria of the genus Roseburia.

The data in the examples show that a preparation of Roseburia alone can offer equal or better protection from cancer progression compared to a mix of different butyrate-producing bacteria previously shown to be reduced in patients with colorectal cancer.

In certain particular embodiments, the bacterial composition for treatment or prevention of recurrence of cancer comprises only bacteria of the genera Anaerostipes, and Roseburia.

In certain particular embodiments, the bacterial composition for treatment or prevention of recurrence of cancer comprises, or in certain embodiments consists of, one or more bacterial species selected from Roseburia intestinalis, Roseburia hominis, Roseburia faecis, Roseburia. inulinivorans, Roseburia cecicola, Anaerostipes caccae, Anaerostipes butyraticus, Anaerostipes rhamnosivorans and/or Anaerostipes hadrus.

In certain particular embodiments, the bacterial composition for treatment or prevention of recurrence of cancer comprises, or in certain embodiments consists of, one or two bacterial species selected from Roseburia intestinalis, Roseburia hominis, Roseburia faecis, Roseburia. inulinivorans, Roseburia cecicola, Anaerostipes caccae, Anaerostipes butyraticus, Anaerostipes rhamnosivorans and/or Anaerostipes hadrus.

In certain particular embodiments, the bacterial composition for treatment or prevention of recurrence of cancer comprises, or in certain embodiments consists of
bacteria of the species Roseburia intestinalis, Roseburia hominis, Roseburia faecis, Roseburia. inulinivorans, and/or Roseburia cecicola, and
bacteria of the species Anaerostipes caccae, Anaerostipes butyraticus, Anaerostipes rhamnosivorans and/or Anaerostipes hadrus.

The data in the examples show that a mix comprising Roseburia intestinalis and Anaerostipes caccae can offer equal or better protection from cancer arising from a range of tissues compared to standard-of-care chemotherapy and immunotherapy antineoplastic agents.

A preparation comprising the bacterial species Anaerostipes hadrus, Anaerostipes butyraticus, Anaerostipes rhamnosivorans are expected to provide similar protection to Anaerostipes caccae, as they are species of the genera Anaerostipes with a similar niche, phenotype, and phylogenic characteristics.

A preparation comprising the bacterial species Roseburia hominis, Roseburia faecis, Roseburia inulinivorans, and/or Roseburia cecicola is expected to provide similar protection to Roseburia intestinalis, as they are species of the genera Roseburia with a similar niche, phenotype, and phylogenic characteristics.

In certain particular embodiments, the bacterial composition for treatment or prevention of recurrence of cancer comprises only bacteria of the species Anaerostipes caccae, in other words, the composition consists of, as far as the bacterial content is concerned, bacteria of the species Anaerostipes caccae.

The data in the examples show that a preparation of Anaerostipes caccae alone can offer equal or better protection from cancer progression in comparison to a mix of different butyrate-producing bacteria previously shown to be reduced in patients with colorectal cancer. A preparation consisting of the bacterial species Anaerostipes hadrus, Anaerostipes butyraticus, or Anaerostipes rhamnosivorans alone is expected to provide similar protection to Anaerostipes caccae, as it is a species of the genera Anaerostipes with a similar niche, phenotype, and phylogenic characteristics.

In another possible embodiment, the bacterial composition for treatment or prevention of recurrence of cancer comprises only bacteria of the species Roseburia intestinalis, in other words, the composition consists of, as far as the bacterial content is concerned, bacteria of the species Roseburia intestinalis.

The data in the examples show that a preparation of Roseburia intestinalis alone can offer equal or better protection from cancer progression in comparison to a mix of different butyrate-producing bacteria previously shown to be reduced in patients with colorectal cancer. A preparation consisting of one of the bacterial species Roseburia hominis, Roseburia faecis, Roseburia inulinivorans, and/or Roseburia cecicola alone is expected to provide similar protection to Roseburia intestinalis, as they are species of the genera Roseburia with a similar niche, phenotype, and phylogenic characteristics.

In certain particular embodiments, the bacterial composition for treatment or prevention of recurrence of cancer comprises, or in certain embodiments consists of, the bacterial strains R. intestinalis DSM14610T, and/or A. caccae DSM14662T.

The data in the examples show that a preparation which comprises, or sometimes consists of, the bacterial strain A. caccae DSM14662T can offer equal or better protection from cancer arising from a range of tissues compared to standard-of-care chemotherapy and immunotherapy antineoplastic agents. A preparation which comprises, or consists of a bacterial strain selected from Anaerostipes butyraticus DSM22094, Anaerostipes rhamnosivorans DSM26241, A. hadrus DSM108065, and/or DSM3319 is expected to provide similar protection, as they are strains from the genera Anaerostipes with a similar niche, phenotype, and phylogenic characteristics to A. caccae DSM14662T.

The data in the examples show that a preparation which comprises, or sometimes consists of, the bacterial strain R. intestinalis DSM14610T can offer equal or better protection from cancer arising from a range of tissues compared to standard-of-care chemotherapy and immunotherapy antineoplastic agents. A preparation which comprises, or consists of a bacterial strain selected from R. hominis DSM16839, R. faecis DSM16840, R. inulinivorans DSM108070, and/or R. cecicola ATCC33874 is expected to provide similar protection, as they are strains from the genera Roseburia with a similar niche, phenotype, and phylogenic characteristics to R. intestinalis DSM14610T.

Certain embodiments relate to a composition according to the invention comprising, or in certain embodiments consisting of, one or more of the bacterial Anaerostipes and/or Roseburia genera, species, or strain as specified above, having been isolated from a human faecal sample. Another embodiment relates to the bacteria genera, species, or strain according to this invention having been obtained from an environmental sample.

An isolate may be identified as an Anaerostipes and/or Roseburia genus, species, or strain by molecular biology techniques known in the art, for example, evaluation of sequence polymorphisms present in one or more copies of the 16S rRNA, or rpoBI gene. For example, a bacterial isolate may be classified as an Anaerostipes or Roseburia species, or strain as specified in an embodiment of the invention if the 16S rRNA gene sequence of the isolate is determined to have >97% similarity to the 16S rRNA gene sequence of a known Anaerostipes or Roseburia species, or >99% similarity to a known Anaerostipes or Roseburia strain 16S rRNA gene sequence, respectively (Johnson J. S. 2010 Nat. Comm. 10:5029).

In certain embodiments, the bacterial composition according to any of the aspects or embodiments of the invention disclosed herein is used for the treatment of cancer, particularly an epithelial cell-derived cancer. A non-exclusive list of epithelial cell cancers which might benefit from bacterial treatment includes lung, breast, brain, prostate, spleen, pancreatic, biliary tract, cervical, ovarian, head and neck, oesophageal, gastric, liver, skin, kidney, bone, testicular, small intestinal, bladder, colon or rectal cancer (CRC), skin cancer, melanoma or sarcoma. In some embodiments, the CRC has a CpG island methylator phenotype (CIMP). In some embodiments, the CRC is a serrated neoplasia.

While the inventors have demonstrated the prophylactic and therapeutic efficacy of the bacterial compositions described above in pre-clinical mouse models of colon cancer, melanoma, breast cancer and lung cancer, the skilled artisan will recognise that this approach can be applied to a number of other epithelial cell-derived cancer types.

In some embodiments of the current invention, the bacterial composition according to any of the aspects or embodiments of the invention disclosed herein is used for the treatment of a patient is or has previously been diagnosed with a solid cancer. In an alternative embodiment, the patient is considered to be at risk of developing cancer.

In embodiments, the bacterial composition according to any of the aspects or embodiments of the invention disclosed herein is used for the treatment of a patient diagnosed with colorectal cancer (CRC), or non-dysplastic serrated polyps, or serrated crypt foci.

In certain embodiments, the bacterial composition is used for the treatment of a cancer derived from an organ that is not part of the gastrointestinal tract, particularly the bacterial composition is used for the treatment of a cancer selected from lung cancer, breast cancer, or melanoma.

In certain embodiments, the bacterial composition is delivered to a healthy subject with the aim of preventing cancer. In certain particular embodiments, the bacterial composition is delivered to a subject who is considered to have predisposition to cancer due to genetic or environmental risk factors. In a further embodiment, the bacterial composition is administered to a patient who has previously been diagnosed with cancer, in order to prevent the recurrence of disease. In some embodiments, the bacterial composition is administered to a patient within the period of cancer remission following other medical interventions, including, but not limited to, chemotherapy, surgical, or radiation treatment. The period of cancer remission according to the invention can include a period of signs or symptoms of disease, such as reduced tumour growth, or total disappearance of the tumour.

The data in the examples show that a preparation that comprises a consortium, or single-strains of defined Clostridiales bacteria can limit different types of tumour growth when the bacteria is administered either before, or after the onset of cancer.

In certain embodiments, the bacterial composition for treatment or prevention of recurrence of cancer is composed of isolated live bacteria. “Isolated” in this context refers to bacteria which are not part of a faecal transplant but have been produced by separating, or isolating bacteria from a natural source (and optionally, culturing them).

It is envisioned that in particular embodiments, the bacterial strains included in the composition are alive, or in the form of viable spores when they reach the intestine of the subject. The composition may comprise a mix of lyophilised bacteria, or lyophilised single strains which are combined with a pharmaceutically acceptable carrier. It is understood that the bacterial composition may also contain a mixture of live bacteria and a certain percentage of dead bacteria, or non-viable spores.

The data in the examples demonstrates the therapeutic efficacy of Clostridiales strains R. intestinalis and A. caccae administered in the form of live cultures or lyophilised bacteria for use as a treatment to inhibit tumour growth in models of cancer.

In an alternative embodiment, the bacterial composition for treatment or prevention of recurrence of cancer is composed of heat-killed bacteria, or cellular component or metabolites derived from the bacteria described in the invention.

In one particular embodiment, the bacterial composition for treatment or prevention of recurrence of cancer is free of faecal matter. In other words, the composition contains isolated bacterial strains that have been produced in an industrial setting, rather than being isolated or cultured from human faecal samples.

In some embodiments, the subject does not receive any antibacterial agents in preparation for, or within a medically relevant window prior to, administration of the bacterial composition for treatment or prevention of recurrence of cancer (for example, within a month prior to treatment). Alternatively, in other embodiments, the subject is treated with an antibacterial agent in preparation of, and prior to, administering the bacterial composition.

In clinical trials of faecal microbiota transplantation known in the art, antibacterial pre-treatment is commonly used to remove certain bacterial species from the intestine, to provide a colonisation niche for therapeutic bacteria, to supress infections in an immunosuppressed individual, or to treat an infectious disease. Examples of antibiotics that can be administered for these purposes include, but are not limited to, kanamycin, gentamicin, colistin, metronidazole, vancomycin, clindamycin, fidaxomicin, and/or cefoperazone. It is understood that the antibacterial agent may also be delivered concurrently with the bacterial composition.

The data in the examples show that a bacterial composition according to the invention inhibits tumour growth in a model of CRC to a similar extent either with, or without prior treatment with an antibacterial agent.

Another embodiment relates to the use of a bacterial preparation according to the invention, for use in a patient within a medically relevant window prior to, concurrent with, or within a medically relevant window following, administration of a checkpoint inhibitor antibody. In particular embodiments, the enteral administration of the bacterial preparation, together with parenteral administration of a checkpoint inhibitor antibody according to this aspect of the invention is provided for use in a patient who has been diagnosed with a cancer likely to be, or shown to be, resistant to treatment with either medicament alone. In particular embodiments, the bacterial administration is administered to a patient who has, is, or will soon receive, an anti-PD-1, or anti-PD-L1 checkpoint inhibitor antibody.

In one such embodiment, a bacterial preparation according to the invention, and a checkpoint inhibitor antibody as specified above, are provided for use in a patient diagnosed with a tumour characterised by resistance, or lack of response to checkpoint inhibitor antibody treatment. Particular embodiments relate to a bacterial preparation according to the invention, and a checkpoint inhibitor antibody as specified above, for use in a patient diagnosed with a metastatic colon cancer, as this cancer is characterised by resistance to checkpoint inhibitor antibody treatment

In another such embodiment, a bacterial preparation according to the invention, and a checkpoint inhibitor antibody as specified above, are provided for use in a patient diagnosed with a tumour characterised by resistance, or a lack of response to a bacterial composition comprising Roseburia and/or Anaerostipes as specified according to the first aspect of the invention.

The lack of term response in the context of the specification refers to an absence of improvement in one or more clinical parameter following treatment, for example, no decrease in tumour size, rate of growth, or spread. Lack of response encompasses observations based on previous treatment outcomes in the patient or subject, general classifications based on clinical knowledge of a particular tumour type, such as the known poor (<5%) response rate of metastatic colon cancer patients to immunotherapy, or a lack of response as defined by an in-vitro assay using patient tumour cells.

The data in the examples show that oral treatment with a bacterial composition that comprises Roseburia intestinalis and Anaerostipes caccae as part of mix of butyrate-producing strains in combination with injections of an anti-PD1 antibody can provide better protection against colon cancer than anti-PD1 antibody treatment alone. The data in FIG. 8 of the examples shows that a combination medicament comprising both an anti-PD1 antibody together with bacteria can provide an unexpected therapeutic benefit in a model of treatment-resistant melanoma which is not responsive to either treatment alone.

In some embodiments, the bacterial composition is provided for us in a patient whose tumour has been determined to be characterised by a paucity of infiltrating immune cells, such as natural killer (NK) cells, NKT cells, or T cells, particularly cytotoxic CD8+ T cells. A paucity, or absence of immune infiltration may be identified by means known in the art, including, but not limited to immunohistochemical staining with immune markers such as CD3, or CD45, and may encompass samples where immune infiltrate is peritumoural rather than within tumour tissues (Hendry 2017 Adv. Anat. Pathol. 24(6):311). Low numbers of immune infiltration might be defined, for example, as under 1000, or under 500 CD3 positive cells counted on a tumour microarray slide section. In certain embodiments, the bacterial composition is provided for use in such subject in order to increase or induce an immune response to the cancer. In other words, the bacterial composition is provided for use in a patient whose tumour has been determined to by characterised by no, or limited, immune cell infiltration. These immune cells are thought to be those most important for killing tumours cells and thus inhibiting tumour growth and spread.

The data in the examples show that bacterial compositions that comprise, or consist of the bacterial species Roseburia intestinalis and Anaerostipes caccae can increase the numbers and/or the activation status of immune cells. This increased immune response in cancer subjects which received the bacterial composition was observed in both tumour tissues and in lymphoid organs such as the spleen, and was greater than that observed following administration of the standard-of-care antineoplastic drug fluorouracil or immunotherapy.

In one embodiment, the bacterial composition for treatment or prevention of recurrence of cancer is administered without previous, concurrent, or subsequent administration of an antineoplastic cancer treatment. In other words, the subject receives the bacterial composition, without any other additional preventative or therapeutic treatment for cancer. Therapeutic treatment in this sense is understood to encompass checkpoint inhibitory agents, particularly checkpoint inhibitor antibodies, as well as antineoplastic chemotherapeutic agents.

The data in the examples show that treatment with a bacterial composition that comprises

Roseburia intestinalis and Anaerostipes caccae as part of mix of butyrate-producing strains can offer equal inhibition of cancer compared to standard-of-care fluorouracil treatment, and better protection from cancer than immunotherapy antineoplastic agents.

A further set of embodiments of the invention refer to the bacterial composition as part of a combination medicament for use in the treatment or the prevention of recurrence of cancer. It is envisioned that the bacterial composition may be administered at the same time, or overlapping with another antineoplastic treatment. In one particular embodiment, the bacterial composition may be delivered as a component of a combination medicament comprising both a bacterial preparation and an antineoplastic agent or treatment.

In one embodiment, the combination medicament comprises a bacterial preparation according to the invention, and an antineoplastic agent, particularly a cytotoxic chemotherapy selected from 5-Fluoruracil, capecitabine, irinotecan, topotecan, floxuridin, oxaliplatin, carboplatin, cisplatin, gemcitabine, paclitaxel, docetaxel, cyclophosphamide, ifosfamid, trofosfamid, chlorambucil, melphalan, busulfan, carmustin, lomustin, semustin, dacarbazin, mitomycin C, methotrexate, raltitrexed, 6-mercaptopurin, thioguanin, cladribin, fludarabin, vincristine, vindesin, bleomycine, actinomycin D, doxorubicin, daunorubicin, epirubicin, idarubicin, mitoxantrone, etoposide, tenoposide, hydroxyurea, and/or procarbazine.

In one embodiment, the combination medicament comprises a bacterial preparation according to the invention, and a hormone targeting agent, particularly a hormone targeting agent selected from leuprolide, goserelin, letrozole, arimidex, exemestane, tamoxifen, toremifene, fulvestrant, lapatinib, palbociclib, raloxifene, anastrazole, triptorelin, histrelin, degarelix, flutamide, enzalutamide, apalutamide, biculatamide, nilutamide, abiraterone, acetate, ketoconazole, and/or aminoglutethimide.

In a further embodiment, the combination medicament comprises a bacterial preparation according to the invention, and a checkpoint inhibitor antibody, particularly a checkpoint inhibitor antibody selected from anti-PD-1, anti-PD-L1, anti-PD-L2, or anti-CTLA-4.

In yet another embodiment, the combination medicament comprises a bacterial preparation according to the invention, and an adjuvant, cytokine, antibody or antibody-like molecule that activates immune cells, particularly a checkpoint agonist agent. In more particular embodiments, the immune checkpoint agonist agent is selected from the clinically available antibody drugs aldesleukin (Novartis, Cas. No 110942-02-4), interferon alfa-2b (Merck, CAS No. 215647-85-1), imiquimod (apotex, CAS No. 99011-02-6), PF-8600 (Pfizer), poly ICLC (oncovir, CAS No. 59789-29-6), cabiralizumab (apexigen, 1613144-80-1) or utomilumab, (CAS No. 1417318-27-4). For a list of further relevant checkpoint agonist agents in development see Sun H. and Sun C. Front. Immunol. (October 2019).

In one embodiment, the combination medicament comprises a bacterial preparation according to the invention, and a checkpoint agonist antibody, particularly a checkpoint agonist antibody selected from anti-CD40, anti-OX40, anti-LAG-3, anti-TIM3, anti-ICOS, anti-TIGIT, or anti-VISTA.

In another embodiment, the combination medicament comprises a bacterial preparation according to the invention, and an immune cell transfer treatment, particularly transfer of autologous cells selected from chimeric antigen receptor T cells, activated lymphocytes, or activated dendritic cells.

The data in the examples show that treatment with a bacterial composition that comprises Roseburia intestinalis and Anaerostipes caccae together, or as part of mix of bacterial strains, or Roseburia intestinalis alone can enhance aspects of the subject's immune response to tumours. This includes infiltration of CD8+ T cells and NK cells into tumour tissues, correlating with reduced tumour weight or volume. For this reason, it is envisioned that the bacterial composition will be most advantageous in terms of clinical outcome when combined with a therapy that aims to increase a subject's immune response to cancer.

In one embodiment, the combination medicament comprises a bacterial preparation according to the invention, and a surgical intervention.

In one embodiment, the combination medicament comprises a bacterial preparation according to the invention, and a nutritional supplement or prebiotic, particularly a nutritional treatment selected from dietary fibre inulin, oligofructose, and/or oligosaccharides.

Another alternative aspect of the invention relates to a combination medicament comprises a bacterial preparation according to the invention, and radiotherapy.

Similarly, the invention provides for a combination of a bacterial preparation, with a form of gene therapy. Examples of genes used for nucleic acid transfer are tumour supressing genes, or genes that activate a prodrug. In another embodiment the preparation could be delivered alongside a genetically engineered oncolytic virus designed to kill tumour cells. An example is the drug T-VEC (talimogene laherparepvec), also known as Imlygic (Cas No. 1187560-31-1). In some embodiments, recombinant forms of the bacteria may be used that express a tumor specific or tumor-associated antigen, or molecules known to enhance human immune activation are contemplated as a type of gene therapy.

The invention further relates to methods of treatment of cancer, wherein an effective amount of a bacterial composition or a combination medicament as provided herein is administered to a patient in need thereof.

The invention further encompasses a bacterial composition according to any one of aspects specified above, for use in the manufacture of a medicament for the treatment, or the prevention of recurrence of cancer, particularly a solid tumour derived from an epithelial-cell origin.

The invention further encompasses a method of treating a patient diagnosed with cancer, particularly a patient diagnosed with a solid, epithelial-cell derived tumour, or a subject determined to be at risk of developing cancer, for example a patient who has been determined to have a genetic predisposition to cancer, or who has been determined to have a microbiota thought to generate a predisposition to cancer, comprising administering to the patient or subject a therapeutically effective amount of the bacterial composition according to the aspects of the invention provided herein.

Routes of Administration and Dosage

The specific therapeutically effective dose of the bacterial compositions according to the invention level for any particular subject will depend upon a variety of factors including the presence or absence of cancer, the type of cancer being treated, the severity of the cancer, the activity or viability of the specific organism or combined composition, the route of administration, the rate of colonization or clearance of the organism or combined composition, the duration of treatment, the drugs (if any) used in combination with the organism, the age, body weight, sex, diet, and general health of the subject, and similar factors well known in the medical arts and sciences.

The data in the examples show that various bacterial compositions according to the invention inhibited tumour growth in murine cancer models when administered 3 times a week, at dose ranging from 10⁸-10⁹ live bacteria. A therapeutically effective dosage level can be ascertained by the skilled artisan by means of animal models of disease, or by reference to the dosages of live bacteria delivered in human clinical trails through similar routes of administration.

In certain particular embodiments, the bacterial composition according to the invention is formulated for enteral, or topical administration. The term “topical” is understood to mean local administration, in other words applied directly to a tumour tissue. This could be in the form a cream or gel applied to an accessible tumour such as a form of skin cancer, or it could also apply to using an injection to deliver the bacteria composition in solution directly into a solid tumour.

In one set of embodiments, the bacterial composition is formulated for oral delivery into the small or large intestines of the subject, where the majority of the gut microbiota reside. One such embodiment relates to enteric coatings that protect the bacterial composition from high pH in the stomach, and dissolve on reaching the intestines. Examples of such coatings include, without being limited to polymers and copolymers such as eudragit (Evonik).

In a similar embodiment the bacterial composition may be delivered into a specific region of the intestines in the form of buffered sachets, or with a coating that dissolves in a pH range specific to a certain portion of this intestine. For example, a formulation which decomposes in the pH range from 6.8 to 7.5, will favour delivery to the colon (for a full description of targeted delivery to regions of the gastrointestinal tract see Villena et al 2015. Int J. Pharm. 487 (1-2):314-9.)

In yet another similar embodiment, the bacterial composition can be administered specifically to the intestines by means of a time-delay delivery method, which takes into account the time it takes to transit through the stomach, small intestine and colon. Delayed release formulations include hydrogel preparations, and biodegradable, water-soluble, hydrolysable or enzyme degradable polymers. Examples of coating materials that are suitable for delayed-release formulations include, but are not limited to, cellulose-based polymers, acrylic acid polymers, and vinylpolymers.

In another embodiment where the bacterial composition is formulated for delivery to the colon, the formulation includes a coating which can be removed by an enzyme present in the human gut, for example a carbohydrate reductase. Examples of enzyme-sensitive coatings include amylose, xanthan gum and azoploymers.

In certain embodiments, the bacterial composition can be targeted to a particular site through intubation of an orifice, or with a surgical intervention.

In embodiments of the invention relating to topical uses of the compounds of the invention, the pharmaceutical composition is formulated in a way that is suitable for topical administration such as aqueous solutions, suspensions, ointments, creams, gels or sprayable formulations, e.g., for delivery by aerosol or the like, comprising the active ingredient together with one or more of solubilizers, stabilizers, tonicity enhancing agents, buffers and preservatives that are known to those skilled in the art.

In embodiments of the invention that relate to rectal administration the bacterial composition may be formulated for delivery as a suppository, enema or as part of an endo- or colonoscopy procedure.

Wherever alternatives for single separable features such as, for example, a bacterial species or strain, cancer type or co-administered cancer drug are laid out herein as “embodiments”, it is to be understood that such alternatives may be combined freely to form discrete embodiments of the invention disclosed herein.

The invention is further illustrated by the following examples and figures, from which further embodiments and advantages can be drawn. These examples are meant to illustrate the invention but not to limit its scope.

### DESCRIPTION OF THE FIGURES

Time course analyses show the mean±the standard error of the mean (SEM) analysed by two-way analysis of variance (ANOVA), with Dunnett's post-test correction for multiple comparisons. Bar graphs indicate the mean±SEM analysed by Mann-Whitney test for comparison of two groups, or the Kruskal-Wallis test with Dunn's correction for multiple comparison. *P<0.05, **P<0.01, *“P<0.001, **”P<0.001.

FIG. 1 shows treatment with a 4-mix of Clostridiales bacteria induces MC38 colon cancer tumour shrinkage in the C57BI6 mouse model with or without antibiotics treatment. (A) Experimental setting: Oral supplementation with 4-mix was performed in C57BI6 mice for 3 days prior subcutaneous injection of MC38 cells, with or without pre-treatment with antibiotics (Abx). Oral gavage with bacteria was repeated one and two weeks later for 3 consecutive days each time. (B) Quantification of subcutaneous MC38 tumour size measured in C57BI6 mice orally gavaged with saline or 4-mix during the course of the experiment. Bar charts showing the (C) tumour and (D) spleen weight of control and Bp treated mice on day 17. Data are representative of two or more independent experiments.

FIG. 2 shows treatment with a 4-mix of Clostridiales bacteria induces MC38 colon cancer tumour shrinkage via induction of CD3+ T cells in C57BI6 mice. (A) Experimental design for CD3+ T cells depletion experiment. Anti-CD8 antibody or isotype control was injected intraperitoneally at days −3, 0, 7, and 14 in reference to subcutaneous MC-38 tumour cell injection. (B) Subcutaneous tumour size was measured at the timepoints indicated. (C) Final tumour weight on day 17 after MC38 tumour cell injection and (D) quantification of CD3+ T cells in immunohistochemistry of MC-38 tumour sections. Data are representative of two or more independent experiments.

FIG. 3 shows treatment with a 4-mix of Clostridiales bacteria is more effective than anti-PD1 therapy in the MC38 colon cancer model. (A) Experimental setting for anti-PD1 experiment in control and 4-mix treated mice, with or without intraperitoneal anti-PD-1 injections. (B) Timecourse showing MC-38 subcutaneous tumour volume measured at the days indicated. (C) Bar chart showing MC-38 tumour volume and (D) weight was measured at day 16. Data are representative of two or more independent experiments.

FIG. 4 shows treatment with a 4-mix of single Clostridiales bacterial species induces MC38 colon cancer tumour shrinkage. (A) Experimental setting: Oral supplementation with 4-mix was performed in C57BI6 mice for 3 days prior subcutaneous injection of MC-38 cells. Oral gavage was repeated one and two weeks later for 3 consecutive days. (B) Timecourse showing MC-38 subcutaneous tumour volume measured at the days indicated. (C) Bar charts showing the tumour volume and (D) tumour weight measured on sacrifice at day 17. Data are representative of two or more independent experiments.

FIG. 5 shows treatment with a 4-mix or single Clostridiales bacterial species exhibits therapeutic potential in the MC38 colon cancer model. Oral supplementation with 4-mix or the species indicated was performed in C57BI6 mice for 3 starting 6 days post-subcutaneous injection of MC-38 cells. Oral gavage was repeated one week later for 3 consecutive days. (A) Timecourse showing MC-38 subcutaneous tumour volume measured at the days indicated, and table indicating statistical comparisons analysed by two-way ANOVA with Dunnett's post-test. (B) Bar charts showing the tumour weight measured on sacrifice at day 15. Data are representative of two or more independent experiments.

FIG. 6 shows the efficacy of treatment with a 4-mix of Clostridiales bacteria or Roseburia intestinalis is equivalent to fluorouracil (5-FU) in the MC38 colon cancer model. C57BI6 mice received oral gavage with PBS, 4-mix of butyrate-producing Clostridiales (BP) or R. intestinalis for 3 days at day 6 and 12 after subcutaneous injection of MC38 tumour cells. The indicated groups also received i.p injections of 50 mg/kg of the standard-of-care chemotherapy 5-FU in PBS at days 6, 9, and 12. (A) Timecourse showing MC-38 subcutaneous tumour volume measured at the days indicated. (B) Bar chart showing the tumour weight measured on sacrifice at day 15. Data are representative of two or more independent experiments.

FIG. 7 shows enhanced antitumour immune responses in spleen following combination treatment with a 4-mix Clostridiales bacterial species compared to 5-FU. Oral supplementation with 4-mix or the species indicated was performed in C571316 mice for 3 starting 6 days post-subcutaneous injection of MC-38 cells. Oral gavage was repeated one week later for 3 consecutive days as in FIG. 9. The indicated groups also received i.p injections of 20 mg/kg of the standard-of-care chemotherapy 5-FU in PBS at days 6, 9, and 12. Bar charts show the proportion of cells positive for the immune activation markers indicated, measured by flow cytometry of tumour infiltrating lymphocytes at day 15 of tumour growth. Data are representative of two or more independent experiments.

FIG. 8 shows treatment with a 4-mix or single Clostridiales bacterial species exhibits therapeutic potential in the B16 melanoma model. (A) Timecourse of B16 tumour growth in C571316 mice. 200 mg of anti-PD-1 or a control IgG was administered to the indicated groups at day 6, 9, and 12 after subcutaneous injection of B16 melanoma cells. (B) Timecourse of tumour volume measured in C57BI6 mice receiving oral gavage with PBS, a 4-mix of Clostridiales or the indicated Clostridiales species for 3 days at day 6 and 12 after subcutaneous injection of B16 tumour cells. Table summarises the results of ANOVA with Dunnet's post-test shown in B. Data are representative of two or more independent experiments.

FIG. 9 shows treatment with a 4-mix Clostridiales consortium (CC) exhibits therapeutic potential in the 4T1 breast cancer model and the LLC1.1 lung cancer model.

Timecourse of tumour volume measured in C57BI6 mice receiving oral gavage with PBS, or a 4-mix of at day 6 and 12 after subcutaneous injection of (A) 4T1 breast cancer cells or (B) LLC2 lung cancer cells. Data are representative of two or more independent experiments.

FIG. 10 shows treatment with a 2-mix of reconstituted lyophilised R. intestinalis+A. caccae inhibits the MC38 colon cancer in a C57BI6 mouse model compared to a PBS control group. Oral supplementation was performed every 3 days, starting 6 days post-subcutaneous injection of MC-38 cells. Timecourse showing MC-38 subcutaneous tumour volume measured at the days indicated.

### EXAMPLES

Methods

Mice

WT C57BL/6JRJ were purchased from Janvier Labs (France). All mice were kept in specific-pathogen-free conditions. Males and female littermates between 8-12 weeks were used for all the experiments.

Bacteria Culture and Treatment

Eubacterium hallii: DSM3353T, Faecalibacterium prausnitzii: DSM17677, Roseburia intestinalis: DSM14610T, Anaerostipes caccae: DSM14662T were obtained from PharmaBiome AG, Zürich. All strains were cultivated at 37° C. in yeast extract, casitone and fatty acid (YCFA) medium (Lopez et al. 2012. Appl Environ Microbiol 78, 420-428). Strains were maintained under anoxic CO2 atmosphere using Hungate techniques. The mix of butyrate-producing bacteria was prepared using equal volumes of two-day cultures of each bacterial strain. Purity of bacteria was confirmed by Gram stain microscopy and analysis of their metabolite production using High-Performance-Liquid-Chromatography. Viability of cells was confirmed using propidium iodide staining measured by flow cytometry. Bacteria were administered by oral gavage in a concentration of 10⁸-10⁹ live bacteria/ml in 200 microliter volume. 0.9% NaCl was used as placebo.

Lyophilized Bacteria Treatment

Lyophilized R. intestinalis (10⁶ cells/g) and A. caccae (10⁹ cells/g) were equally mixed. 1 g of bacteria was reconstituted in 1 ml of water for 2-3 minutes before supplemented to the mice by oral gavage. Each mouse received 200 pl of the bacteria mix. Control mice received 200 pl water by oral gavage.

Antibiotic Pre-Treatment

One week prior starting bacteria treatment, mice received drinking water supplemented with 1 g/L neomycin, 0.5 g/L vancomycin, 1 g/L ampicillin, 0.2% (w/v) aspartam for 7 days, in addition to daily gavage for a week with the antibiotics plus 1 g/L metronidazole. After the antibiotic treatment and one day before starting the bacteria treatment, mice received 10% polietilenglicol (PEG) 3000 in the drinking water overnight.

Tumour Models and Treatments

Tumour cell lines were maintained in Dulbecco's modified Eagle's medium supplemented with 100 U/ml penicillin/streptomycin and 10% heat-inactivated fetal calf serum (FCS) at 37 ° C. in 5% CO2. For subcutaneous tumour models, MC38-GFP colorectal cancer (3×10⁵ cells, kindly provided by Prof. Lubor Borsig, University of Zürich), B16-GFP melanoma cells (3×10⁵ cells, kindly provided by Prof. Onur Boyman, University Hospital Zürich) or Lewis lung carcinoma cells LLC1.1 (2×10⁵ cells, ATCC No. CRL-1642) were suspended in DMEM high glucose cell culture medium mixed 1:1 with matrigel and injected subcutaneously into the flanks. Tumour development was measured every 3 days using a digital calibrator. Tumour volume was calculated using the ellipsoid formula: 4/3*3.14*Length/2*(Width/2)², where the shorter dimension was used as width and depth. Mice were euthanized when the volume reached 1 cm³ or the length reached 2 cm. For therapeutic administration of bacteria, mice were treated with bacterial mix per by oral gavage for three consecutive days starting at day 5 and day 12. Mice were terminated on day 21. For the 4T1 breast cancer model, mice were treated with bacterial mix by oral gavage for three days starting at day −2 and on a third day (day 0), breast cancer cells 4T1 (100,000 cells, ATCC No. CRL-2539) dissolved in a matrigel were injected in the mammary fat pad of a mouse. Mice were treated with bacterial mix per gavage for three consecutive days starting at day 5 and day 12. Mice were terminated on day 21.

CD3+ T cells depletion was performed in the subcutaneous injection model using anti-CD8 (Lyt 3.2) antibody (BioXCell; clone 53-5.8) or IgG isotype control (BioXCell; clone HRPN). Antibodies were injected i.p 200 mg/mouse on day −3, and 100 mg/mouse on day 0, 7 and 14. PD-1 blockade was performed by injecting of 200 mg/mouse anti-PD1(CD279) antibody (BioXCell; clone 29F.1Al2) or IgG isotype control (BioXCell; clone 2a3) intraperitoneally on days 6, 9 and 12 after subcutaneous tumour cell injection.

Flow Cytometry

Single cell suspensions from spleen, mesenteric and skin draining lymph nodes, colon lamina propia lymphocytes (LPL), and tumour cells were used for flow cytometry analysis. Cecum and subcutaneous tumours were cut to approximately 0.5 mm³ pieces and digested in 6 mL containing 0.5 mg/mL collagenase type IV (Sigma Aldrich) and 0.05 mg/mL DNAse I (Roche) solution for 10 minutes on a shaker (300 rpm) at 37° C. Cells were homogenized passing through 18G1.5 syringe and centrifuged for 10 mins, 4° C., 1500 rpm. Single cell suspensions were stained and re-stimulated for 4 h with 10 ug/ml Brefeldin A (SIGMA) as described previously (Spalinger et al. 2019, Mucosal Immunol. 12, 1336-1347).

Statistical Analysis

When comparing two groups, non-parametric two-tailed Mann Whitney test was used. For comparisons between three or more groups, ANOVA, or non-parametric two-tailed Kruskal-Wallis test was used and Dunn's post-hoc test applied.

### Example 1

Oral application of a 4-mix of Clostridiales bacteria inhibits colon cancer The faecal bacterial constitution of 768 CRC patients from five study sites (Wirbel et al. Nat. Med. 25 (4):1, 2019) was mapped and compared to the microbial signatures of healthy controls. Populations across five geographical areas each demonstrated differential signatures between patients and healthy controls but had remarkable similarities between the geographically different patient groups. Faecal shotgun metagenomics studies identified that out of the ten strains which were significantly more represented in healthy controls compared to CRC patients across the cohorts, eight were from the Clostridiales family. Based on this analysis, four Clostridiales strains were selected, namely Roseburia intestinalis, Eubacterium hallii (Anaerobutyricum hallii), Faecalibacterium prausnitzii, and Anaerostipes caccae (4-mix of bacteria) for experimentational investigations in murine models of CRC to determine whether manipulating Clostridiales can prevent tumour growth. Both the 4-mix and the individual strains Roseburia intestinalis and Anaerostipes caccae showed considerable efficacy in both prophylactic and therapeutic models of cancer treatment as outlined below.

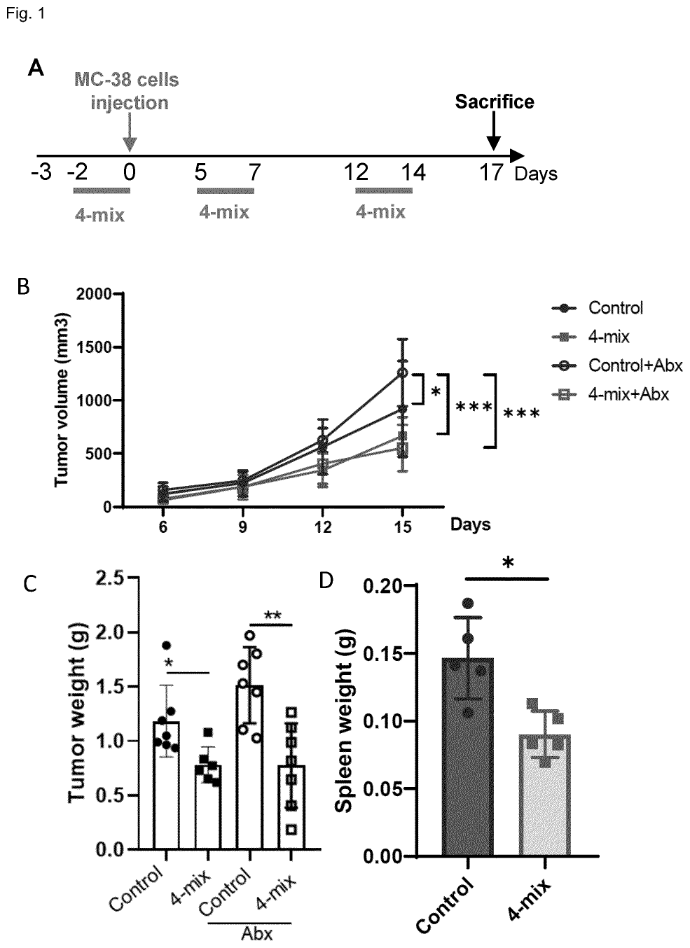

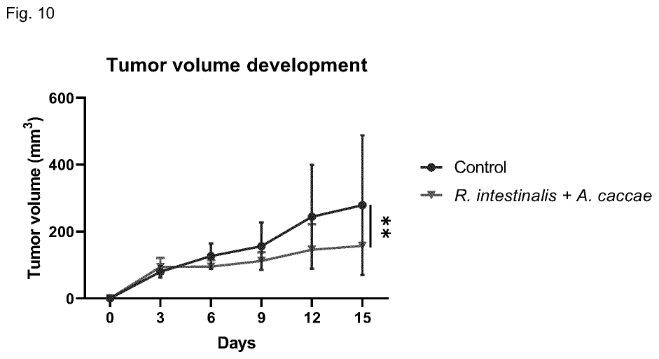

C57BI6 mice were treated with 4-mix and injected subcutaneously with MC-38 mouse CRC cells (FIG. 1A). Oral supplementation with 4-mix resulted in significantly reduced tumour growth in terms of tumour volume (FIG. 1B) and weight (FIG. 10). A reduced spleen weight in treated mice indicated no significant systemic inflammation or infection was induced by the Clostridales 4-mix regime (FIG. 1 D).

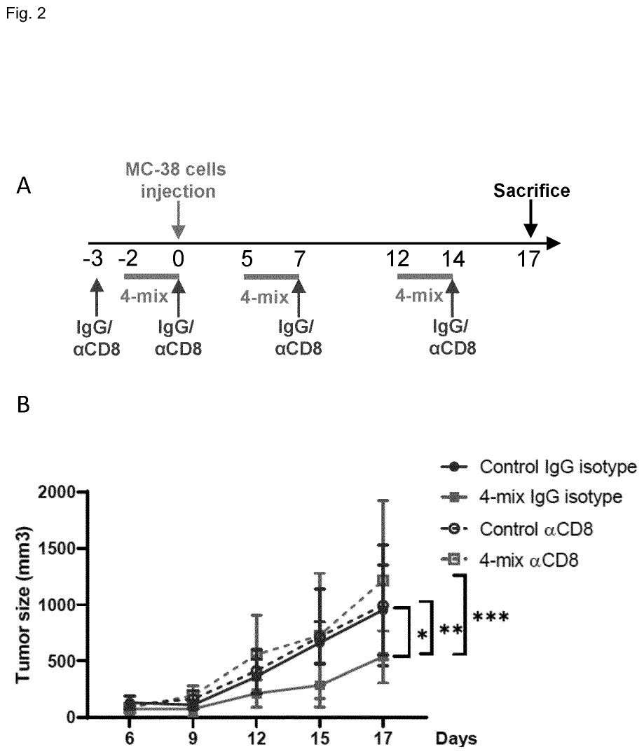

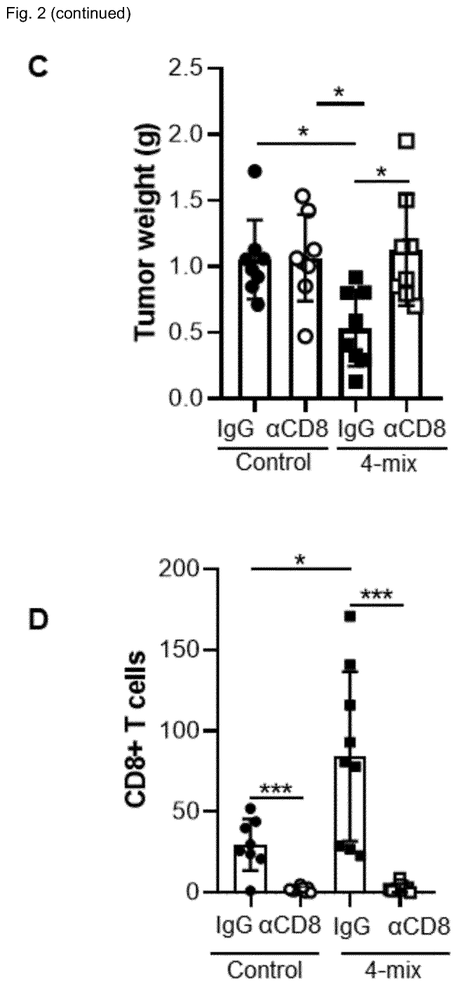

4-mix of Clostridiales bacteria activates CD3+ T cells to inhibit tumour growth Cytotoxic CD3+ T cells are an important mediator of antitumour immunity. The antitumour effect of the 4-mix was completely abrogated upon antibody-mediated CD3+ T cell depletion (FIG. 2A-C). Additionally, infiltration of CD3+ T cells was significantly higher in mice treated with 4-mix compared to controls (FIG. 2D). Together, these data indicate that the 4-mix exerts a systemic anti-tumour effect by increasing the infiltration of CD8+ T cells and cytotoxic antitumour CD8+ T cell responses.

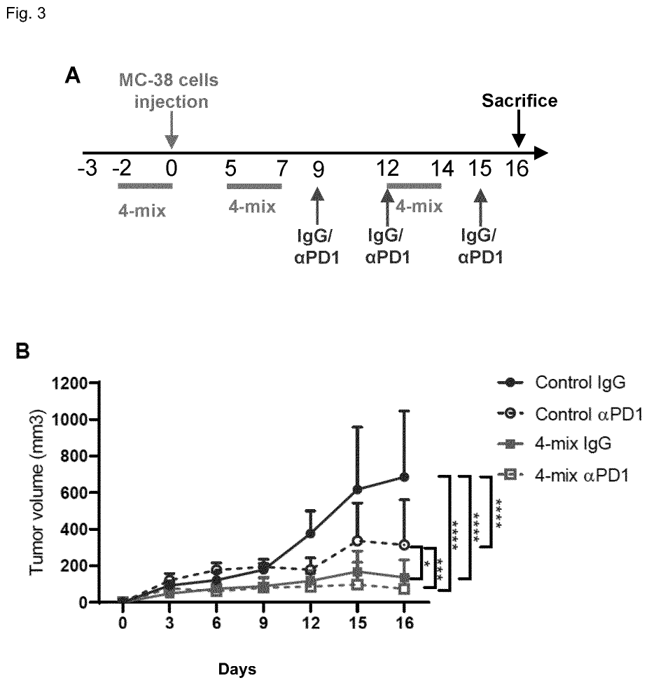

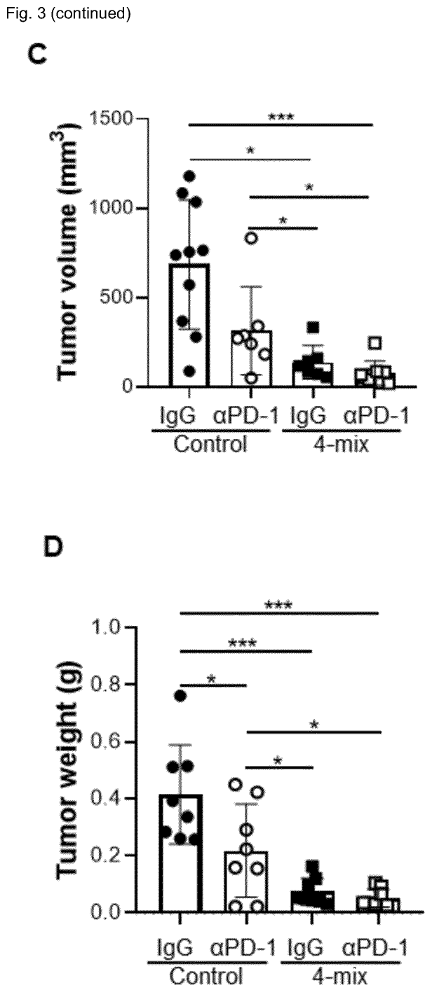

Oral Clostridiales bacteria is more effective that anti-PD-1 therapy Ineffectiveness of immune checkpoint blockade immunotherapy in CRC is often due to poor T cell infiltration into the tumours. As the 4-mix increases the infiltration of CD8+ T cells into the tumour tissue, a therapeutic approach of anti-PD-1 treatment in combination with our 4-mix consortium was tested for synergistic effects (FIG. 3A). Anti-PD-1 therapy showed only marginal anti-tumour efficacy in our model system CRC model, as in most patients with this disease (FIG. 3B-D). In contrast, 4-mix treatment significantly reduced tumour growth compared to anti-PD-1 treated mice, suggesting that in this model treatment with 4-mix is more efficient that anti-PD-1 immunotherapy (FIG. 3B-D). Combination of 4-mix and anti-PD-1 did not show additional anti-tumour benefit over the 4-mix alone (FIG. 3B-D).

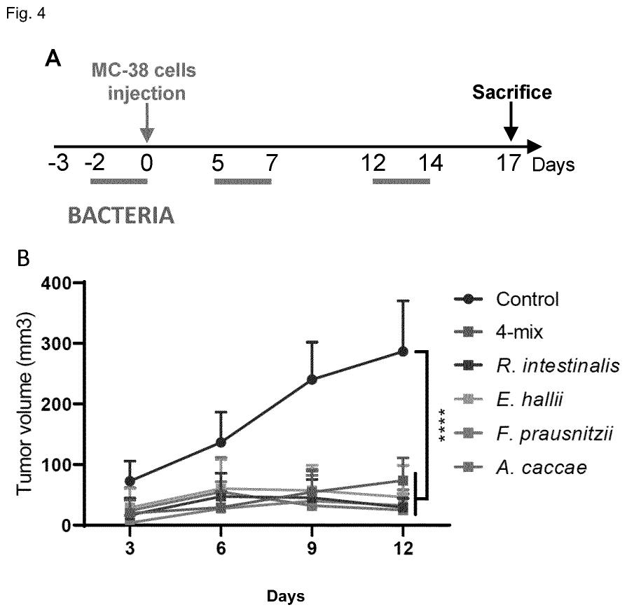

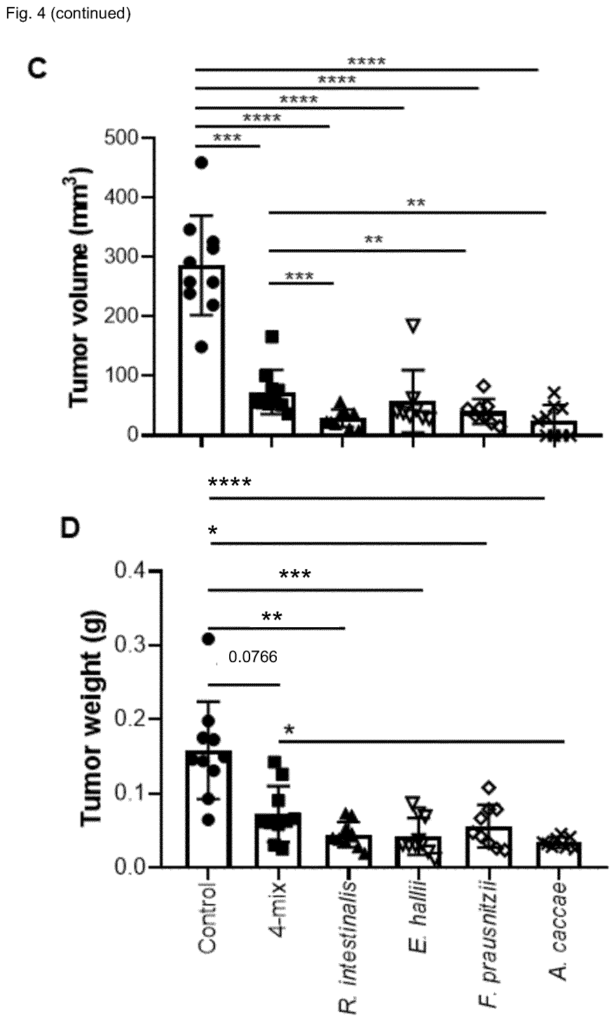

As bacterial consortia pose manufacturing and regulatory challenges in development for clinical applications, the anti-tumour efficacy of single strains of our bacteria consortium were assessed compared to the 4-mix (FIG. 4A). Individually, the single strains were at least as efficient as the 4-mix consortium or presented an even stronger anti-tumour effect, particularly for R. intestinalis, and A. caccae for both, tumour volume (FIG. 4B and C) and tumour weight (FIG. 4D).

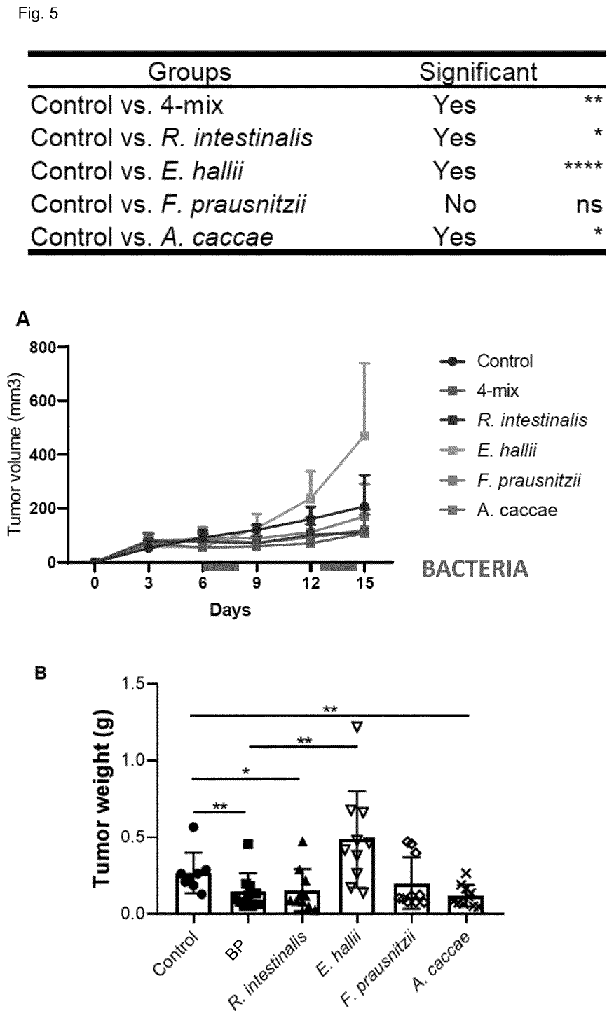

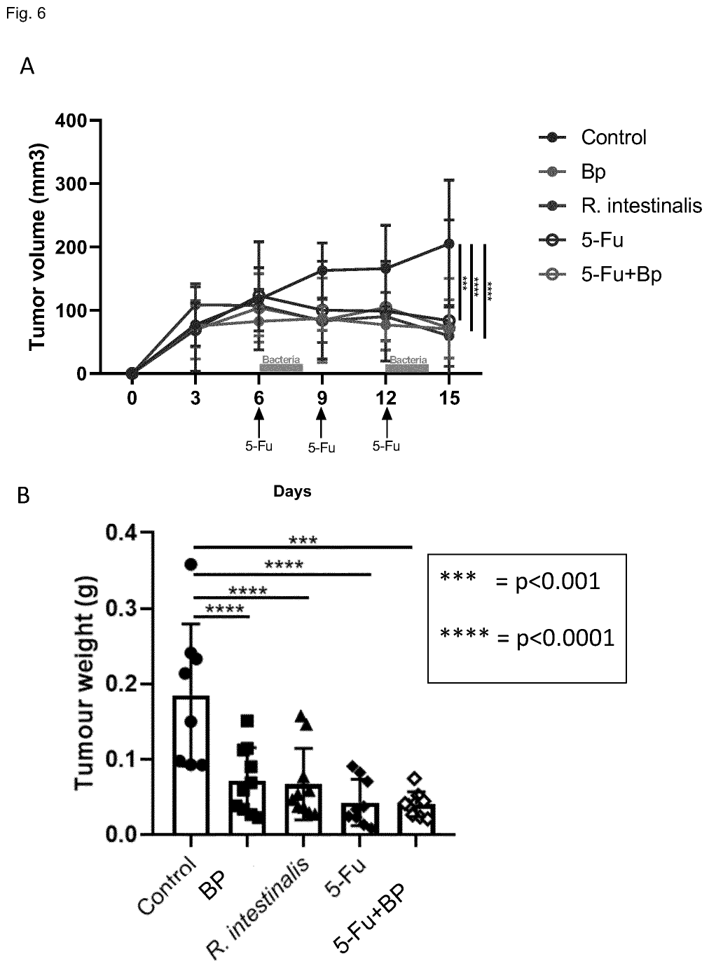

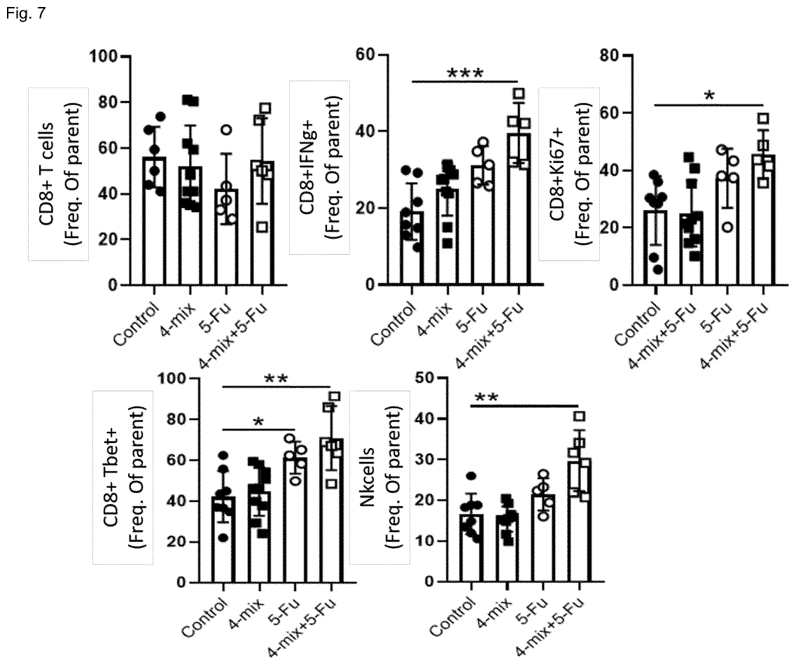

Oral Clostridiales bacteria boosts immune response against established cancer While causality is difficult to demonstrate in human data, murine models allow investigations into possible therapeutic effects of reintroducing helpful bacteria into the intestine once a tumour is established. Indeed, the 4-mix as well as R. intestinalis and A. caccae were successful in the therapeutic treatment of mice with CRC tumours (FIG. 5A and B). To provide further clinical context, the efficacy of oral 4-mix was compared with the clinical standard-of-care chemotherapeutic agent 5-fluorouracil (5-FU). The 4-mix treatment reduced tumour growth to a similar extent as 5-FU (FIG. 6A). Combination of 4-mix and 5-FU further increased the frequencies of IFN-γ, Ki67, and T-bet within CD8+ T cells, and NK cells in tumour tissue (FIG. 7). These results demonstrate that 4-mix is as efficient as the clinical standard of care chemotherapy with 5-FU in our experimental context.

Oral Clostridiales bacteria therapy inhibits the growth of tumours from diverse tissues

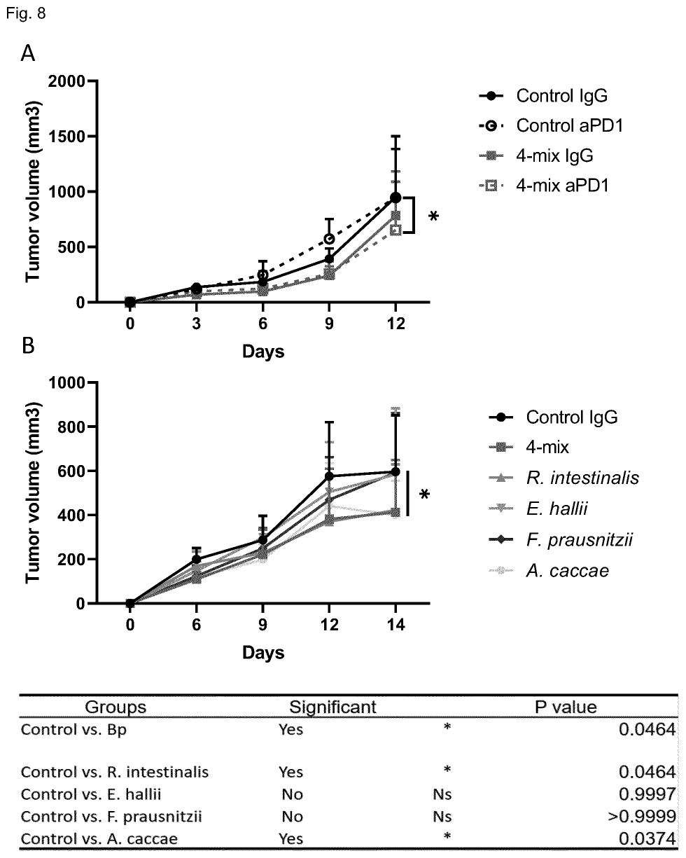

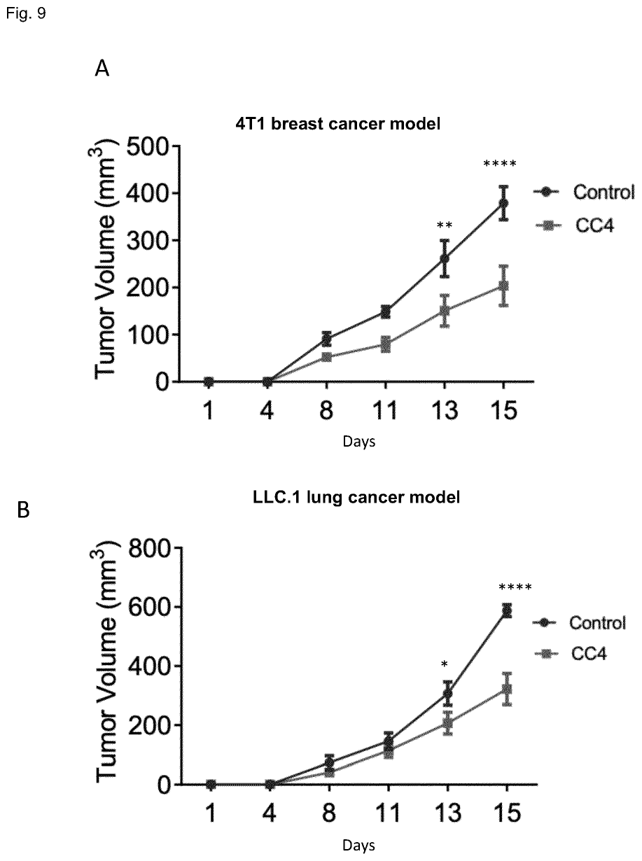

To determine whether oral Clostridiales treatment has universal anti-tumour capacity, the 4-mix and single strains was tested in the immunotherapy-resistant B16 melanoma model, with or without anti-PD1 antibody. Administration of 4-mix in combination with anti-PD1 reduced the size of B16 tumours (FIG. 8A). When testing single strains, mice treated with R. intestinalis and A. caccae together with the 4-mix showed significantly reduced B16 tumours compared with control mice (FIG. 8B). Further tests in the 4T1 breast cancer model (FIG. 9A) and the LLC1.1 lung cancer model (FIG. 9B) found that anti-tumour efficacy of 4-mix was comparable to results obtained in the CRC and melanoma models. These data identify a universal anti-tumour mechanism of oral bacterial treatment, in both CRC and a broad range of solid tumours, in both a prophylactic and therapeutic setting showing they may be promising therapy in cancers resistant to immunotherapy treatment. Finally, the MC38 model was used to confirm that reconstituted lyophilised mixture of the Clostridiales strains R. intestinalis and A. caccae were able to inhibit tumour growth in a model of cancer (FIG. 10).

### SUMMARY

Oral bacteria therapy with the above strains drive an enhanced anti-tumour immune response by means of increased CD8+ T cell infiltration into tumour tissue, characterised by production of IFN-γ, and decreased expression of immune checkpoint inhibitors. Bacteria are effective prophylactically, which might be a driver of homeostatic protection against CRC and may prove applicable to populations with a strong genetic predisposition or a family history of CRC. It is also promising as therapeutic approach when tumours have already been established. These preclinical findings demonstrate the feasibility of novel cancer therapy based solely on gut microbiota supplementation as stand-alone therapy.

1. A bacterial composition only comprising, or consisting of, one or more of the genera of bacteria selected from:
Anaerostipes, and/or
Roseburia,
for use in treatment, or for prevention of recurrence, of cancer.

2. The bacterial composition for use in treatment or prevention of recurrence of cancer according to claim 1, wherein the bacterial composition only comprises, or consists of, bacteria of the genus Anaerostipes.

3. The bacterial composition for use in treatment or prevention of recurrence of cancer according to claim 1, wherein the bacterial composition only comprises, or consists of, bacteria of the genus Roseburia.

4. The bacterial composition for use in treatment or prevention of recurrence of cancer according to claim 1, wherein the bacterial composition comprises the two single genera of bacteria:
Anaerostipes, and
Roseburia.

5. The bacterial composition for use in treatment or prevention of recurrence of cancer according to claim 1, wherein the bacterial composition comprises or consists of bacteria selected from:
Roseburia intestinalis,
Roseburia hominis,
Roseburia faecis,
Roseburia. inulinivorans,
Roseburia cecicola,
Anaerostipes caccae,
Anaerostipes butyraticus,
Anaerostipes rhamnosivorans and
Anaerostipes hadrus.

6. The bacterial composition for use in treatment or prevention of recurrence of cancer according to claim 1, wherein the bacterial composition comprises or consists of a single, or two bacterial species selected from:
Roseburia intestinalis,
Roseburia hominis,
Roseburia faecis,
Roseburia. inulinivorans,
Roseburia cecicola,
Anaerostipes caccae, and
Anaerostipes hadrus.

7. The bacterial composition for use in treatment or prevention of recurrence of cancer according to claim 1, wherein the cancer is a solid tumour, particularly an epithelial cell-derived tumour, more particularly a cancer selected from lung, breast, brain, prostate, spleen, pancreatic, biliary tract, cervical, ovarian, head and neck, oesophageal, gastric, liver, skin, kidney, bone, testicular, small intestinal, colon, or bladder cancer, melanoma or non-melanoma skin cancer or sarcoma.

8. The bacterial composition for use in treatment or prevention of recurrence of cancer according to claim 1, wherein the cancer originates in a tissue that is not the colon or rectum, particularly wherein the cancer is selected from:
lung cancer,
breast cancer, or
melanoma.

9. The bacterial composition for use in treatment or prevention of recurrence of cancer according to claim 1, wherein the bacterial composition is composed of isolated live or lyophilized bacteria, heat-killed bacteria, or isolated bacterial spores, particularly wherein the composition is free of faecal matter.

10. The bacterial composition for use in treatment or prevention of recurrence of cancer according to claim 1, wherein the bacterial composition is administered to a patient not having received an antibacterial agent prior to administering the bacterial composition.

11. The bacterial composition for use in treatment or prevention of recurrence of cancer according to claim 1, wherein the bacterial composition is provided to a patient diagnosed with a solid, malignant tumour characterized by a paucity or absence of tumour infiltrating immune cells, particularly natural killer cells and T cells.

12. A bacterial composition for use in treatment of cancer as in claim 1 that is formulated for enteral, or topical administration.

13. The bacterial composition for use in treatment or prevention of recurrence of cancer according to claim 1, wherein the bacterial composition is administered to a patient prior to, concurrent with, or subsequent to administration of a checkpoint inhibitor antibody, particularly an anti-PD-1, anti-PD-L1 checkpoint inhibitor antibody.

14. The bacterial composition for use in treatment or prevention of recurrence of cancer according to claim 13, for use in a patient diagnosed with a tumour characterised by
a. a lack of response to a bacterial composition and/or
b. a lack of response to a checkpoint inhibitor antibody, particularly an anti-PD-1 or anti-PD-L1 checkpoint inhibitor antibody.

15. The bacterial composition for use in treatment or prevention of recurrence of cancer according to claim 1, wherein the bacterial composition is administered without concurrent, previous, or subsequent administration of a cancer immunotherapy, particularly a checkpoint inhibitor antibody.

16. A combination medicament for use in the treatment or the prevention of recurrence of cancer comprising a bacterial composition as specified in claim 1 and an antineoplastic treatment, particularly a combination medicament comprising the bacterial composition and
a cytotoxic chemotherapy, particularly a cytotoxic chemotherapy selected from 5-Fluoruracil, capecitabine, irinotecan, topotecan, floxuridin, oxaliplatin, carboplatin, cisplatin, gemcitabine, paclitaxel, docetaxel, cyclophosphamide, ifosfamid, trofosfamid, chlorambucil, melphalan, busulfan, carmustin, lomustin, semustin, dacarbazin, mitomycin C, methotrexate, raltitrexed, 6-mercaptopurin, thioguanin, cladribin, fludarabin, vincristine, vindesin, bleomycine, actinomycin D, doxorubicin, daunorubicin, epirubicin, idarubicin, mitoxantrone, etoposide, tenoposide, hydroxyurea, or procarbazin,
a hormone targeting agent, particularly a hormone targeting agent selected from leuprolide, goserelin, letrozole, arimidex, exemestane, tamoxifen, toremifene, fulvestrant, lapatinib, palbociclib, raloxifene, anastrazole, triptorelin, histrelin, degarelix, flutamide, enzalutamide, apalutamide, biculatamide, nilutamide, abiraterone, acetate, ketoconazole, or aminoglutethimide,
a checkpoint inhibitor antibody, particularly a checkpoint inhibitor antibody selected from anti-PD-1, anti-PD-L1, anti-PD-L2, or anti-CTLA-4,
a checkpoint agonist agent, particularly a checkpoint agonist selected from aldesleukin, interferon alfa-2b, imiquimod, PF-8600, Poly ICLC, cabiralizumab, or utomilumab,
a checkpoint agonist antibody, particularly a checkpoint agonist antibody selected from anti-CD40, anti-OX40, anti-LAG-3, anti-TIM3, anti-ICOS, anti-TIGIT, or anti-VISTA,
an immune cell transfer treatment, particularly transfer of autologous cells selected from chimeric antigen receptor T cells, activated lymphocytes, or activated dendritic cells,
a surgical intervention,
a nutritional treatment or prebiotic, particularly a nutritional treatment selected from dietary fibre inulin, oligofructose, and/or oligosaccharides,
radiotherapy,
a gene therapy selected from gene transfer genes transferred could be tumour supressing genes, or genes that activate a prodrug, or a genetically engineered oncolytic virus.
